(19) United States
(12) Patent Application Publication
(10) Pub. No.: US 2025/0171392 A1
(43) Pub. Date: May 29, 2025

(54) CRYSTALLIZATION OF 4-HYDROXYACETOPHENONE FROM ETHANOL AND ETHYL ACETATE

(71) Applicant: SYMRISE AG, Holzminden (DE)

(72) Inventors: Ravikumar PILLAI, Mahwah, NJ (US); Sven SIEGEL, Höxter (DE); Sabrina BEHNKE, Pompton Lakes, NJ (US); Jasmin SALMEN, Detmold (DE); Nikolas BUGDAHN, Holzminden (DE); Jörg NIEKERKEN, Holzminden (DE); Michael MICHLER, Eimen (DE)

(73) Assignee: SYMRISE AG, Holzminden (DE)

(21) Appl. No.: 18/842,440

(22) PCT Filed: Mar. 7, 2023
(86) PCT No.: PCT/EP2023/055719

§ 371 Date: Aug. 29, 2024

(30) Foreign Application Priority Data
Mar. 7, 2022 (WO) PCT/EP2022/055713

(51) Int. Cl. C07C 45/81; C07C 45/79

(52) U.S. Cl. CPC C07C 45/81; C07C 45/79

(57) ABSTRACT

The present invention primarily relates to a method of purifying crude 4-hydroxyacetophenone with a combination of ethanol and ethyl acetate as described herein. The present invention furthermore relates to a product comprising or consisting of crystallized 4-hydroxyacetophenone and ethanol and ethyl acetate, the product being obtained or obtainable by a method as defined herein. Finally, the present invention relates to the use of a combination of ethanol and ethyl acetate to (re) crystallize 4-hydroxyacetophenone.

The present invention primarily relates to a method of purifying crude 4-hydroxyacetophenone with a combination of ethanol and ethyl acetate as described herein. The present invention furthermore relates to a product comprising or consisting of crystallized 4-hydroxyacetophenone and ethanol and ethyl acetate, the product being obtained or obtainable by a method as defined herein. Finally, the present invention relates to the use of a combination of ethanol and ethyl acetate to (re) crystallize 4-hydroxyacetophenone.

Further aspects of the present invention will arise from the description below, in particular from the examples, as well as from the attached patent claims.

4-Hydroxyacetophenone can be used as a pharmaceutical intermediate in the synthesis of drugs such as Paracetamol, Ractopamine and Atenolol. It can also be used as a raw material for cosmetics, and was, for example, embodied in the Used Cosmetics Raw Material Catalogue by the China Food and Drug Administration (CFDA).

When used in cosmetics, 4-hydroxyacetophenone has the following advantageous characteristics:
(a) It has a certain anticorrosion ability,
(b) it has great ability to kill Aspergillus brasiliensis, and
(c) it has a certain ability to restrain Pseudomonas aeruginosa (Daily Chemical Industry: 2015, 45:269).

The standard synthesis of 4-hydroxyacetophenone starts from phenol, which is converted to phenyl acetate by esterification reaction. Phenyl acetate is then converted into 4-hydroxyacetophenone in a rearrangement reaction.

The commercial grade material has a strong odor and color and high phenol content of 100 ppm to 1000 ppm making it unsuitable for cosmetic applications without further purification.

The purification of crude 4-hydroxyacetophenone is usually carried out by means of one of the following methods:
(a) Recrystallization from water (as disclosed in CN102093189A)
(b) Purification by column chromatography (as disclosed in J. Org. Chem., 2011, 76 (7): 2296-2300).

The drawbacks of using purification method (a), i.e. using water as the solvent in the (re)crystallization step, are that the used amount of water is very large (usually about 15 to 20 times of the quantity of the raw material) and the solubility of the crude 4-hydroxyacetophenone in water is low, resulting in a low working efficiency for this method. Moreover, (re)crystallization of 4-hydroxyacetophenone from water usually results in a powder, not a crystallized product, which has bad flowability, is easy to cake and inconvenient to use. If the water is used repeatedly, the content of salt and phenols contained therein will increase continuously. This results in a negative effect on the product quality and high costs for this post-treatment method. Lastly, the color of the final product is darker and thus less attractive to the customer or consumer if small amounts of residual water remain inside the final product.

The drawbacks of using purification method (b), i.e. column chromatography, is that the sample volume that can be purified is limited because of the column size. Thus, this method only is suitable when the sample quantity is small. Moreover, this chromatographic method is quite involved and the solvent volume used is large. Thus, the associated costs are very high and this method is not suitable for industrialization.

Consequently, both of the purification methods described above are not suitable for application on industrial scale, because of the high costs associated with them and their low production efficiency.

Therefore, it was an object of the present invention to provide a purification method for crude 4-hydroxyacetophenone that yields a highly pure, crystalline, colorless and odorless solid with superior solubility in water or cosmetic solvent systems, superior flowability properties, and a very low residual content of the precursor phenol. It was a further object of the invention to provide a purification method for 4-hydroxyacetophenone that is easy to carry out, safe and reliable and highly efficient in terms of solvent consumption and production costs and thus is applicable to production of highly pure 4-hydroxyacetophenone on an industrial scale. Moreover, it was an object of the present invention to provide highly pure, crystalline, colorless and odorless 4-hydroxyacetophenone with superior solubility in water or cosmetic solvent systems and superior flowability properties.

Further objects underlying the present invention follow from the description below and the present patent claims.

According to a first aspect of the present invention, the stated objects are achieved by a method of purifying crude 4-hydroxyacetophenone, comprising or consisting of the following steps:
(a) Providing crude 4-hydroxyacetophenone,
(b) mixing the crude 4-hydroxyacetophenone of step (a) with ethanol and ethyl acetate,
(c) optionally, heating the mixture obtained in step (b) to (fully) dissolve the 4-hydroxyacetophenone,
(d) optionally, adding an adsorbent, preferably activated carbon, to the mixture obtained in step (b) or step (c), if present, (or in any other preceding step),
(e) optionally, cooling the mixture obtained in step (b), step (c) or step (d), if present, to a temperature above the crystallization temperature of 4-hydroxyacetophenone,
(f) if step (d) is present, removing the adsorbent from the mixture of step (d) or step (e), if present, preferably by filtration,
(g) cooling of the mixture obtained in step (b) or step (c), if present, or further cooling of the mixture obtained in step (e), if step (d) is not present, or step (f), if present, to a temperature below the crystallization temperature of 4-hydroxyacetophenone to induce crystallization of 4-hydroxyacetophenone,
(h) collecting the crystallized 4-hydroxyacetophenone obtained in step (g),
optionally, carrying out the additional steps (i) to (k) once or several times:
   (i) dissolving the crystallized 4-hydroxyacetophenone obtained in step (h) or a previous step (k), respectively, in ethanol and ethyl acetate, optionally under heating,
   (j) cooling of the solution of step (i) to a temperature below the crystallization temperature of 4-hydroxyacetophenone to induce crystallization of 4-hydroxyacetophenone,
   (k) collecting the crystallized 4-hydroxyacetophenone obtained in step (j),
(l) optionally, drying of the crystallized 4-hydroxyacetophenone obtained in step (h) or step (k), preferably until the total amount of the residual ethanol and ethyl acetate in the 4-hydroxyacetophenone is less than 10000 ppm, preferably 5000 ppm, preferably less than 2500 ppm, more preferably less than 1000 ppm.

In the context of the own studies underlying the present invention, it was surprisingly found that if the method as defined above is used to purify crude 4-hydroxyacetophenone, highly pure and crystalline 4-hydroxyacetophenone with a purity of at least 90% (by weight), 99.3%, 99.4%, 99.5%, 99.6%, 99.7%, 99.8%, 99.90%, 99.91%, 99.92%, 99.93%, 99.94%, 99.95%, 99.96%, 99.97%, 99.98% or 99.99% (by weight), and with superior properties in terms of (whiter) color, less or more pleasant odor, better flowability and/or with a phenol content of less than 100 ppm (i.e. the amount of the residual phenol being less than 100 ppm) is obtained.

Furthermore, it was surprisingly found that the method according to the invention, i.e. (re)crystallization of 4-hydroxyacetophenone from a combination of the solvents ethanol and ethyl acetate, leads to superior solubility of the obtained 4-hydroxyacetophenone in water, aqueous solutions and other cosmetic solvent systems (as demonstrated in the examples further below).

Thus, the method as defined above is particularly advantageous since the product (crystallized 4-hydroxyacetophenone) obtained or obtainable by the method as defined above is suitable for and easier to use in cosmetic applications. In particular, the much faster dissolution speed of the obtained product in water, aqueous solutions and other cosmetic solvent systems is advantageous since it speeds up the cosmetic formulation process, which will be highly appreciated by formulators. Dissolving cosmetic ingredients in a quick manner and without addition of heat makes it possible to generate sustainable formulations and to save time and costs.

Suitable adsorbents that may be added in step (d) of the method as defined above, preferably for decolorization and/or deodorization, are, for example, activated carbon or molecular sieves.

A preferred embodiment according to the invention is a method as defined above, wherein in step (h) the collection of the crystallized 4-hydroxyacetophenone obtained in step (g) is carried out by means of centrifugation and/or decantation and/or filtration.

Another preferred embodiment according to the invention is a method as defined above, wherein in step (i) after dissolving the crystallized 4-hydroxyacetophenone obtained in step (h) or a previous step (k), respectively, in ethanol and ethyl acetate, optionally under heating, any residual insoluble impurities are removed, preferably by filtration.

Another preferred embodiment according to the invention is a method as defined above, wherein in step (k) the collection of the crystallized 4-hydroxyacetophenone obtained in step (j) is carried out by means of centrifugation and/or decantation and/or filtration.

Preferably the crystallized 4-hydroxyacetophenone obtained after the centrifugation and/or decantation and/or filtration carried out in step (h) and/or (k) is washed with a small amount of a combination of ethanol and ethyl acetate and collected by means of an additional step of centrifugation and/or decantation and/or filtration. More preferably, the filtrates obtained in step (h) and/or (k) after collecting the 4-hydroxyacetophenone are combined and subjected to another cooling and (re)crystallization step to achieve maximal product yield.

In the context of the present invention, the method as defined above necessarily comprises or consists of the steps (a), (b), (g) and (h), and optionally additionally comprises steps (c) and/or (d) and/or (e) and/or (f) and/or (i) and/or (j) and/or (k) and/or (l). If step (d), the addition of the adsorbent, is comprised in the method as defined above, then step (f), the removal of the adsorbent, necessarily has to be present. Preferably, the method as defined above comprises or consists of all of the steps (a) to (h). More preferably, the method as defined above comprises or consists of all of the steps (a) to (h) and step (l).

A further preferred embodiment according to the invention is a method as defined above, comprising or consisting of all of the steps (a) to (I), wherein steps (i) to (k) are carried out once or preferably twice, three, four or more times.

Preferably, and as far as technically sensible, the order of the (optional) method steps following step (b) of the method according to the invention is not necessarily limited to the order specified in the claims. For example, cooling of the mixture obtained in step (c), if present, to a temperature above the crystallization temperature of 4-hydroxyacetophenone (i.e. step (e), if present), can also take place before the addition of an adsorbent (i.e. step (d), if present). Thus, according to a preferred embodiment, the method of purifying crude 4-hydroxyacetophenone according to the invention comprises or consists of the following steps:
(a) Providing crude 4-hydroxyacetophenone,
(b) mixing the crude 4-hydroxyacetophenone of step (a) with ethanol and ethyl acetate,
(c) heating the mixture obtained in step (b) to dissolve the 4-hydroxyacetophenone,
(d) cooling the mixture obtained in step (c) to a temperature above the crystallization temperature of 4-hydroxyacetophenone,
(e) adding an adsorbent, preferably activated carbon, to the mixture obtained in step step (d),
(f) removing the adsorbent from the mixture of step (e), preferably by filtration,
(g) (further) cooling of the mixture obtained in step (f) to a temperature below the crystallization temperature of 4-hydroxyacetophenone to induce crystallization of 4-hydroxyacetophenone,
(h) collecting the crystallized 4-hydroxyacetophenone obtained in step (g),
   optionally, carrying out the additional steps (i) to (k) once or several times:
      (i) dissolving the crystallized 4-hydroxyacetophenone obtained in step (h) or a previous step (k), respectively, in ethanol and ethyl acetate, optionally under heating,
      (j) cooling of the solution of step (i) to a temperature below the crystallization temperature of 4-hydroxyacetophenone to induce crystallization of 4-hydroxyacetophenone,
      (k) collecting the crystallized 4-hydroxyacetophenone obtained in step (j),
(l) optionally, drying of the crystallized 4-hydroxyacetophenone obtained in step (h) or step (k), preferably until the total amount of the residual ethanol and ethyl acetate in the 4-hydroxyacetophenone is less than 10000 ppm, preferably 5000 ppm, preferably less than 2500 ppm, more preferably less than 1000 ppm.

The use of a combination of the solvents ethanol and ethyl acetate in the method as defined above is particularly advantageous, since it enables the purification method to be carried out at gentle temperatures and improves the flowability of the solution of 4-hydroxyacetophenone as well as the flowability and solubility of the crystallized 4-hydroxyacetophenone. Following the method as defined above, the combination of the solvents ethanol and ethyl acetate can be recovered and used repeatedly, which increases production effectiveness and reduces the costs of the purification procedure, and generally of the production procedure, of highly pure 4-hydroxyacetophenone. Since the method as defined above is safe, reliable, cost and material efficient, it is suitable for operation on an industrial scale.

Preferably, the combination of ethanol and ethyl acetate used or formed in steps (b) and/or (i), if present, independently is 0.01 to 90 wt.-%, preferably 0.01 to 80 wt.-%, more preferably 0.01 to 70 wt.-%, more preferably 0.01 to 60 wt.-%, more preferably 0.01 to 50 wt.-%, more preferably 0.1 to 25 wt.-%, more preferably 0.1 to 14 wt.-%, more preferably 0.5 to 14 wt.-%, more preferably 4 to 14 wt. %, more preferably 5 to 12 wt. %, more preferably 5 to 10 wt. %, most preferably 6 to 8 wt. %, of ethanol in ethyl acetate.

In steps (b) and (i), if present, of the method according to the invention, ethanol and ethyl acetate can be added to the (crude) 4-hydroxyacetophenone individually to form a combination of ethanol and ethyl acetate or they can be added to the crude 4-hydroxyacetophenone as a pre-mixed combination of ethanol and ethyl acetate. Alternatively, the (crude) 4-hydroxyacetophenone can be added to one of ethanol and ethyl acetate and then the other can be added afterwards or the (crude) 4-hydroxyacetophenone can be added to a pre-mixed combination of ethanol and ethyl acetate. In all of said scenarios, a mixture of (crude) 4-hydroxyacetophenone, ethanol and ethyl acetate is obtained at the end of steps (b) and (i), if present.

According to a preferred embodiment, the combination of ethanol and ethyl acetate used or formed in step (b) is 0.5, 1, 2, 3, 4, 5, 6, 7, 8, 9, or 10 wt.-% of ethanol in ethyl acetate or any ranges of wt. % of ethanol in ethyl acetate in between said numbers.

According to another preferred embodiment, the combination of ethanol and ethyl acetate used or formed in step (i), if present, is 0.5, 1, 2, 3, 4, 5, 6, 7, 8, 9, or 10 wt.-% of ethanol in ethyl acetate or any ranges of wt. % of ethanol in ethyl acetate in between said numbers.

A method according to the invention is preferred, wherein in step(s) (b) and/or (i), if present, independently 0.1 to 80 wt. %, preferably 0.5 to 70 wt. %, preferably 1 to 60 wt. %, more preferably 5 to 50 wt. %, of 4-hydroxyacetophenone, in each case based on the total weight of the mixture or solution obtained in step (b) or step (i), respectively, are combined with the ethanol and ethyl acetate.

Advantageously, 4-hydroxyacetophenone is mixed with the ethanol and ethyl acetate in an amount such that good solubility of the 4-hydroxyacetophenone in the obtained mixture is achieved, preferably at room temperature and/or when the mixture is heated.

Another preferred embodiment according to the invention is a method as defined herein, wherein in step (c), if present, the mixture obtained in step (b) is heated to a temperature of 30° C. to reflux temperature, preferably of 40° C. to reflux temperature, more preferably of 50° C. to reflux temperature, most preferably of 60 to 70° C., and preferably is kept at said temperature for 1 min to 2 hours, more preferably for 30 min to 1 hour.

Heating the mixture obtained in step (b) to reflux in step (c) is particularly advantageous, because it ensures that all of the 4-hydroxyacetophenone contained in the mixture is fully dissolved, which supports efficient purification of the material.

Another preferred embodiment according to the invention is a method as defined herein, wherein in step (d), if present, 0.1 to 25 wt. %, preferably 0.1 to 10 wt. %, more preferably 0.5 to 5 wt. %, of the adsorbent, based on the total weight of the mixture obtained in step (d), are added to the mixture obtained in step (b) or step (c), if present, (or in any other preceding step).

Advantageously, a sufficiently high amount of adsorbent, preferably of activated carbon, is added to achieve complete decolorization and/or deodorization of the 4-hydroxyacetophenone to be purified.

According to another preferred embodiment of the method according to the invention, the optional steps (d) and (f) are not present. Such embodiment is preferred since it simplifies the method according to the invention, which in turn leads to lower production costs.

Another preferred embodiment according to the invention is a method as defined herein, wherein in case step (c) is present, in step (e), if present, the mixture obtained in step (c) or step (d), if present, is cooled to a temperature of 25 to 75° C., preferably to a temperature of 30 to 75° C., preferably to a temperature of 40 to 70° C., more preferably to a temperature of 55 to 65° C.

If the mixture has been heated in step (c), especially if it has been heated to reflux, it is advantageous to cool down the mixture to a temperature above the crystallization point of the 4-hydroxyacetophenone in step (e), if present, for further handling such as for example for the removal of the adsorbent in step (f), if present. It is important to ensure that the temperature is kept above the crystallization point of 4-hydroxyacetophenone at this point (steps (e) and (f) of the method as defined above, if present) to avoid any losses of product yield.

Another preferred embodiment according to the invention is a method as defined herein, wherein in step (g) the mixture obtained in step (b) or step (c), if present, or the mixture obtained in step (e), if step (d) is not present, or in step (f), if present, is cooled to a temperature of −10° C. to below room temperature, preferably to a temperature of 0 to 20° C., more preferably to a temperature of 5 to 10° C.

The crystallization temperature of the 4-hydroxyacetophenone is dependent on the concentration of the 4-hydroxyacetophenone in the mixture as well as on the concentrations of the ethanol and ethyl acetate and other contaminants, if applicable, in the mixture. The person skilled in the art will lower the temperature of the mixture appropriately until crystallization of 4-hydroxyacetophenone is observed.

Preferably, the cooling rate in step (g) of the method according to the invention is 5 to 50° C. per hour, preferably 10 to 30° C. per hour, most preferably is 25° C. per hour.

Preferably, the cooling rate in step (j) of the method according to the invention, if present, is 5 to 50° C. per hour, preferably 10 to 30° C. per hour, most preferably is 25° C. per hour.

Another preferred embodiment according to the invention is a method as defined herein, wherein in step (j), if present, the solution obtained in step (i), if present, is cooled to a temperature of −10° C. to below room temperature, preferably to a temperature of 0 to 20° C., more preferably to a temperature of 5 to 10° C.

Another preferred embodiment according to the invention is a method as defined herein, wherein the drying of the crystallized 4-hydroxyacetophenone in step (I), if present, is carried out at reduced pressure, preferably at a reduced pressure of 0.1 to 100 mbar, more preferably of 1 to 50 mbar, most preferably of 5 to 10 mbar.

The drying process may be carried out with the aid of, for example, a rotary evaporator, a filter dryer, a conical vertical dryer, a helix dryer, or a horizontal dryer.

Preferably, the drying of the crystallized 4-hydroxyacetophenone in step (I), if present, is carried out at a temperature of 50 to 100° C., preferably of 60 to 90° C., more preferably of 60 to 80° C.

Preferably, the drying time in step (I), if present, is between 1 and 48 hours, preferably 2 and 24 hours, more preferably is about 4 to 20 hours.

Following a drying method as defined herein in step (I) of the method according to the invention is particularly advantageous, since it ensures good flowability and solubility of the final product and avoids caking of the final product.

Another aspect of the present invention relates to a product comprising or consisting of crystallized 4-hydroxyacetophenone and ethanol and ethyl acetate, the product being obtained or obtainable by a method according to the invention as defined herein.

The product obtained or obtainable by a method as defined herein is particularly crystalline, colorless, odorless and/or pure, preferably has a purity of at least 90% (by weight), more 99.3%, 99.4%, 99.5%, 99.6%, 99.7%, 99.8%, 99.90%, 99.91%, 99.92%, 99.93%, 99.94%, 99.95%, 99.96%, 99.97%, 99.98% or 99.99% (by weight), and/or has superior flowability.

Moreover, it was surprisingly found during the studies underlying the present invention that the product obtained or obtainable by a method as defined herein is particularly well soluble in water, aqueous solutions and other cosmetic solvent systems (as will be demonstrated in the examples further below). Thus, the product obtained or obtainable by the method as defined herein is suitable for and easier to use in cosmetic applications. In particular, the much faster dissolution speed of the obtained product in water, aqueous solutions and other cosmetic solvent systems is advantageous since it speeds up the cosmetic formulation process, which will be highly appreciated by formulators since it saves time and costs.

The product obtained or obtainable by a method as defined herein preferably (also) has a phenol content of less than 100 ppm, based on the total weight of the product. Thus, the product obtained or obtainable as defined herein is particularly advantageous since it is suitable for and easier to use in cosmetic applications.

Preferably, the total concentration of the ethanol and ethyl acetate contained in the product is less than 10000 ppm, preferably less than 5000 ppm, preferably less than 2500 ppm, most preferably less than 1000 ppm, respectively, based on the total weight of the product.

When the 4-hydroxyacetophenone is purified and dried according to the method as defined herein, particularly when it is dried according to step (I) of the method as defined herein, the residual total concentration of ethanol and ethyl acetate contained in the purified 4-hydroxyacetophenone can be minimized to less than 10000 ppm, preferably to less than 5000 ppm, preferably to less than 2500 ppm, most preferably to less than 1000 ppm, based on the total weight of the product, thus leading to a product with a purity of at least 90% (by weight), more preferably of at least 91%, 92%, 93%, 94%, 95%, 96%, 97%, 98%, 99.0%, 99.1%, 99.2%, 99.3%, 99.4%, 99.5%, 99.6%, 99.7%, 99.8%, 99.90%, 99.91%, 99.92%, 99.93%, 99.94%, 99.95%, 99.96%, 99.97%, 99.98% or 99.99% (by weight).

Reducing the amount of residual solvent(s) in the purified 4-hydroxyacetophenone is particularly advantageous, since it minimizes the chemical and/or technical odor of the product and yields a particularly colourless product, thus making it more appealing to and particularly safe to use for the customer.

Another aspect of the present invention relates to the use of a combination of ethanol and ethyl acetate to (re) crystallize 4-hydroxyacetophenone.

Preferably, a combination of the solvents ethanol and ethyl acetate is used to (re) crystallize crude 4-hydroxyacetophenone.

Within the framework of the present text, crude 4-hydroxyacetophenone preferably is 4-hydroxyacetophenone that was obtained by synthesis and has not yet been purified.

According to another embodiment, a combination of the solvents ethanol and ethyl acetate is used to (re) crystallize pre-purified 4-hydroxyacetophenone.

Pre-purification can take place by (re)crystallization, i.e. by carrying out steps (a) to (h)—if present—of the method as defined herein. Alternatively, pre-purification of 4-hydroxyacetophenone can also be carried out, for example, by extraction, (column) chromatography, filtration, and/or high vacuum distillation, preferably molecular distillation.

According to a preferred embodiment of the use according to the invention, the combination of ethanol and ethyl acetate is 0.01 to 90 wt.-%, preferably 0.01 to 80 wt.-%, more preferably 0.01 to 70 wt.-%, more preferably 0.01 to 60 wt.-%, more preferably 0.01 to 50 wt.-%, more preferably 0.1 to 25 wt.-%, more preferably 0.1 to 14 wt.-%, more preferably 0.5 to 14 wt.-%, more preferably 4 to 14 wt. %, more preferably 5 to 12 wt. %, more preferably 5 to 10 wt. %, most preferably 6 to 8 wt. %, of ethanol in ethyl acetate.

Preferably, the purity of the ethanol used within the framework of the present text is over 95%, more preferably is over 96, 97, 98, 99, 99.1, 99.2, 99.3, 99.4, 99.5, 99.6, 99.7, 99.8 or 99.9%. It may thus include trace amounts of impurities, e.g. of stabilizers. Most preferably, ethanol of analytical grade is used to keep the contained impurities to a minimum.

Preferably, the purity of the ethyl acetate used within the framework of the present text is over 95%, more preferably is over 96, 97, 98, 99, 99.1, 99.2, 99.3, 99.4, 99.5, 99.6, 99.7, 99.8 or 99.9%. It may thus include trace amounts of impurities, e.g. of stabilizers. Most preferably, ethyl acetate of analytical grade is used to keep the contained impurities to a minimum.

Preferred embodiments of the product according to the invention correspond to or can be derived from the preferred embodiments of the method according to the invention which are explained above or vice versa.

Preferred embodiments of the use according to the invention correspond to or can be derived from the preferred embodiments of the method according to the invention which are explained above or vice versa.

Preferred embodiments of the product according to the invention correspond to or can be derived from the preferred embodiments of the use according to the invention which are explained above or vice versa.

The invention will now be described in more detail hereinafter with references to the examples. Further aspects of the present invention are disclosed in the accompanying claims.

### EXAMPLES

### 1. Purification Procedures

### Example 1a (According to the Invention)

Step 1:320 g of crude 4-hydroxyacetophenone are dissolved in 480 g of 7 wt. % of ethanol in ethyl acetate, heated to 85° C. and refluxed for 30 minutes. Then the temperature is lowered to 65° C. and 6.5 g of activated carbon are added. Mix for 30 minutes. The activated carbon is removed by filtration. Then the temperature of the filtrate is slowly lowered to 5° C. to induce crystallization of the 4-hydroxyacetophenone, and subsequently the filtrate is centrifuged to collect the crystallized, wet product.

Step 2: To the above wet product 260 g of 7 wt. % of ethanol in ethyl acetate are added and heated to 85° C. to obtain a clear solution. Any undissolved impurities, if applicable, are removed by filtration. The temperature of the filtrate is slowly lowered to 5° C. to induce crystallization of the 4-hydroxyacetophenone, then the filtrate is centrifuged to separate the precipitated 4-hydroxyacetophenone. After removal of the supernatant, a small quantity of 7 wt. % of ethanol in ethyl acetate is used to wash the crystallized 4-hydroxyacetophenone. The collected washing solution and decanted supernatant were combined and used for another crystallization step to obtain maximum product yield.

Step 3: The wet, fine product obtained above was transferred into a rotary evaporator, a vacuum of 0.1-10 mbar was applied, slowly heated to 50-70° C. and the product dried for 4-8 hours. 85 g of the final product in the form of white, crystalline 4-hydroxyacetophenone were obtained. Purity: 99.9%, melting point: 110-111° C.

Similar results were obtained with varying concentrations of ethanol in ethyl acetate.

### Example 1b (According to the Invention)

Step 1:54.5 g of crude 4-hydroxyacetophenone are dissolved in 26.8 g of 7 wt. % of ethanol in ethyl acetate, heated to 78° C. and refluxed for 30 minutes. Then the temperature of the clear solution is slowly lowered to 0° C. A crystal of pure 4-hydroxyacetophenone is added at 57° C. to (further) induce crystallization of the 4-hydroxyacetophenone. After stirring for 15 min at 0° C., the crystals are collected by vacuum filtration.

Step 2: The obtained crystals are washed with 22 g of a cold mixture of EtOH/EtOAc (7:93). The wet, fine product obtained above is transferred into a rotary evaporator, a vacuum of 1 mbar is applied, slowly heated to 50-70° C. and the product dried for 8 hours. 36.4 g of the final product in the form of white, crystalline 4-hydroxyacetophenone is obtained. Purity: >99.9%, melting point: 110-111° C.

### Example 2 (Comparative)

Step 1:250 g of crude 4-hydroxyacetophenone are dissolved in 1500 g of water, heated to 95° C. and refluxed for 30 minutes. Then the temperature is slowly lowered to 5° C. to induce crystallization of the 4-hydroxyacetophenone, and subsequently the filtrate is centrifuged to collect the crystallized, wet product.

Step 2: The wet, fine product obtained above was transferred into a rotary evaporator, a vacuum of 0.1-10 mbar was applied, slowly heated to 50-70° C. and the product dried for 6-8 hours. 233 g of the final product in the form of off-white, crystalline 4-hydroxyacetophenone were obtained. Purity: 99.9%, melting point: 110-111° C.

Similar results were obtained when a second (re)crystallization step was carried out before drying of the product.

### Example 3 (Comparative)

Step 1:250 g of crude 4-hydroxyacetophenone are dissolved in 125 g of methanol, heated to 75° C. and refluxed for 30 minutes. Then the temperature is lowered to 65° C. and 5 g of activated carbon are added. Mix for 30 minutes. The activated carbon is removed by filtration. The filtrate is subsequently brought to crystallization by slowly lowering the temperature to 5° C. to induce crystallization of the 4-hydroxyacetophenone, and subsequently the filtrate is centrifuged to collect the crystallized, wet product.

Step 2: The wet, fine product obtained above was transferred into a rotary evaporator, a vacuum of 0.1-10 mbar was applied, slowly heated to 50-70° C. and the product dried for 6-8 hours. 133 g of the final product in the form of white, crystalline 4-hydroxyacetophenone were obtained. Purity: 99.9%, melting point: 110-111° C.

### Example 4 (Comparative)

Step 1:250 g of crude 4-hydroxyacetophenone are dissolved in 200 g of ethanol, heated to 85° C. and refluxed for 30 minutes. Then the temperature is lowered to 65° C. and 6.5 g of activated carbon are added. Mix for 30 minutes. The activated carbon is removed by filtration. Then the temperature of the filtrate is slowly lowered to 5° C. to induce crystallization of the 4-hydroxyacetophenone, and subsequently the filtrate is centrifuged to collect the crystallized, wet product.

Step 2: To the above wet product 125 g of ethanol are added and heated to 85° C. to obtain a clear solution. Any undissolved impurities, if applicable, are removed by filtration. The temperature of the filtrate is slowly lowered to 5° C. to induce crystallization of the 4-hydroxyacetophenone, then the filtrate is centrifuged to separate the precipitated 4-hydroxyacetophenone. After removal of the supernatant, a small quantity of ethanol is used to wash the crystallized 4-hydroxyacetophenone. The collected washing solution and decanted supernatant were combined and used for another crystallization step to obtain maximum product yield.

Step 3: The wet, fine product obtained above was transferred into a rotary evaporator, a vacuum of 0.1-10 mbar was applied, slowly heated to 50-70° C. and the product dried for 4-8 hours. 76 g of the final product in the form of white, crystalline 4-hydroxyacetophenone were obtained. Purity: 99.9%, melting point: 110-111° C.

### Example 5 (Comparative)

Step 1:250 g of crude 4-hydroxyacetophenone are dissolved in 500 g of diethyl carbonate, heated to 85° C. and refluxed for 30 minutes. Then the temperature is lowered to 65° C. and 5 g of activated carbon are added. Mix for 30 minutes. The activated carbon is removed by filtration. Then the temperature of the filtrate is slowly lowered to 5° C. to induce crystallization of the 4-hydroxyacetophenone, and subsequently the filtrate is centrifuged to collect the crystallized, wet product.

Step 2: To the above wet product 350 g of diethyl carbonate are added and heated to 70° C. to obtain a clear solution. Any undissolved impurities, if applicable, are removed by filtration. The temperature of the filtrate is slowly lowered to 5° C. to induce crystallization of the 4-hydroxyacetophenone, then the filtrate is centrifuged to separate the precipitated 4-hydroxyacetophenone. After removal of the supernatant, a small quantity of diethyl carbonate is used to wash the crystallized 4-hydroxyacetophenone. The collected washing solution and decanted supernatant were combined and used for another crystallization step to obtain maximum product yield.

Step 3: The wet, fine product obtained above was transferred into a rotary evaporator, a vacuum of 0.1-10 mbar was applied, slowly heated to 50-70° C. and the product dried for 6-8 hours. 157 g of the final product in the form of white, crystalline 4-hydroxyacetophenone were obtained. Purity: 99.9%, melting point: 110-111° C.

### Example 6 (Comparative)

Step 1:250 g of crude 4-hydroxyacetophenone are dissolved in 500 g of dimethyl carbonate, heated to 85° C. and refluxed for 30 minutes. Then the temperature is lowered to 65° C. and 5 g of activated carbon are added. Mix for 30 minutes. The activated carbon is removed by filtration. Then the temperature of the filtrate is slowly lowered to 5° C. to induce crystallization of the 4-hydroxyacetophenone, and subsequently the filtrate is centrifuged to collect the crystallized, wet product.

Step 2: To the above wet product 350 g of dimethyl carbonate are added and heated to 70° C. to obtain a clear solution. Any undissolved impurities, if applicable, are removed by filtration. The temperature of the filtrate is slowly lowered to 5° C. to induce crystallization of the 4-hydroxyacetophenone, then the filtrate is centrifuged to separate the precipitated 4-hydroxyacetophenone. After removal of the supernatant, a small quantity of dimethyl carbonate is used to wash the crystallized 4-hydroxyacetophenone. The collected washing solution and decanted supernatant were combined and used for another crystallization step to obtain maximum product yield.

Step 3: The wet, fine product obtained above was transferred into a rotary evaporator, a vacuum of 0.1-10 mbar was applied, slowly heated to 50-70° C. and the product dried for 6-8 hours. 150 g of the final product in the form of white, crystalline 4-hydroxyacetophenone were obtained. Purity: 99.9%, melting point: 110-111° C.

### Example 7 (Comparative)

A sample of 4-hydroxyacetophenone (re) crystallized from a mixture of dimethyl carbonate and cyclohexane was obtained from a commercial supplier.

### Example 8 (Comparative)

A sample of 4-hydroxyacetophenone (re) crystallized from a mixture of dimethyl carbonate and ethanol was obtained from a commercial supplier.

### Example 9 (Comparative)

Step 1:320 g of crude 4-hydroxyacetophenone are dissolved in 480 g of 7 wt. % of methanol in ethyl acetate, heated to 85° C. and refluxed for 30 minutes. Then the temperature is lowered to 65° C. and 6.4 g of activated carbon are added. Mix for 30 minutes. The activated carbon is removed by filtration. Then the temperature of the filtrate is slowly lowered to 5° C. to induce crystallization of the 4-hydroxyacetophenone, and subsequently the filtrate is centrifuged to collect the crystallized, wet product.

Step 2: To the above wet product 230 g of 7 wt. % of methanol in ethyl acetate are added and heated to 85° C. to obtain a clear solution. Any undissolved impurities, if applicable, are removed by filtration. The temperature of the filtrate is slowly lowered to 5° C. to induce crystallization of the 4-hydroxyacetophenone, then the filtrate is centrifuged to separate the precipitated 4-hydroxyacetophenone. After removal of the supernatant, a small quantity of 7 wt. % of methanol in ethyl acetate is used to wash the crystallized 4-hydroxyacetophenone. The collected washing solution and decanted supernatant were combined and used for another crystallization step to obtain maximum product yield.

Step 3: The wet, fine product obtained above was transferred into a rotary evaporator, a vacuum of 0.1-10 mbar was applied, slowly heated to 50-70° C. and the product dried for 4-8 hours. 65 g of the final product in the form of white, crystalline 4-hydroxyacetophenone were obtained. Purity: 99.9%, melting point: 110-111° C.

### Example 10 (Comparative)

Step 1:250 g of crude 4-hydroxyacetophenone are dissolved in 500 g of ethyl acetate, heated to 77° C. and refluxed for 30 minutes. Then the temperature is lowered to 65° C. and 5 g of activated carbon are added. The mixture is stirred at 77° C. for 30 minutes. The activated carbon is removed by filtration at 70° C. Then the temperature of the filtrate is slowly lowered over 3 h to 5° C. A crystal of pure 4-hydroxyacetophenone is added at 40° C. to (further) induce crystallization of the 4-hydroxyacetophenone. The suspension is stirred another hour at 5° C. before collecting 146 g crystals by vacuum filtration.

Step 2: To the above wet product 250 g of ethyl acetate are added and heated to reflux to obtain a clear solution. The temperature of the solution is slowly lowered over 3 h from 50 to 5° C. A crystal of pure 4-hydroxyacetophenone is added at 38° C. to (further) induce crystallization of the 4-hydroxyacetophenone. After another hour stirring at 0° C., the crystals are collected by vacuum filtration. After removal of the supernatant, a small quantity of ethyl acetate (25 g) is used to wash the crystallized 4-hydroxyacetophenone.

Step 3: The wet, fine product obtained above is transferred into a rotary evaporator, a vacuum of 0.15 mbar is applied, slowly heated to 50° C. and the product dried for 6 hours. 77.3 g of the final product in the form of white, crystalline 4-hydroxyacetophenone is obtained. Purity: >99.9%, melting point: 110-111° C.

The obtained samples of purified 4-hydroxyacetophenone were analyzed with regard to their physical appearance and their solubility in water and selected aqueous solutions, respectively (as shown below).

Within the framework of the present study, 4-hydroxyacetophenone was (re) crystallized from a multitude of different solvents and solvent mixtures under different conditions. The analysis of the obtained purified 4-hydroxyacetophenone samples confirmed that 4-hydroxyacetophenone obtained with the method according to the invention has an advantageous physical appearance (cf. Table 1 below) and surprisingly displayed by far the shortest dissolution time in water and aqueous solutions (cf. Tables 2-4 below). Only a selected number of examples and comparative examples are included in the present text for the sake of efficiency.

### 2 Physical Appearance of the Different Samples of Purified 4-Hydroxyacetophenone (4-HAP)

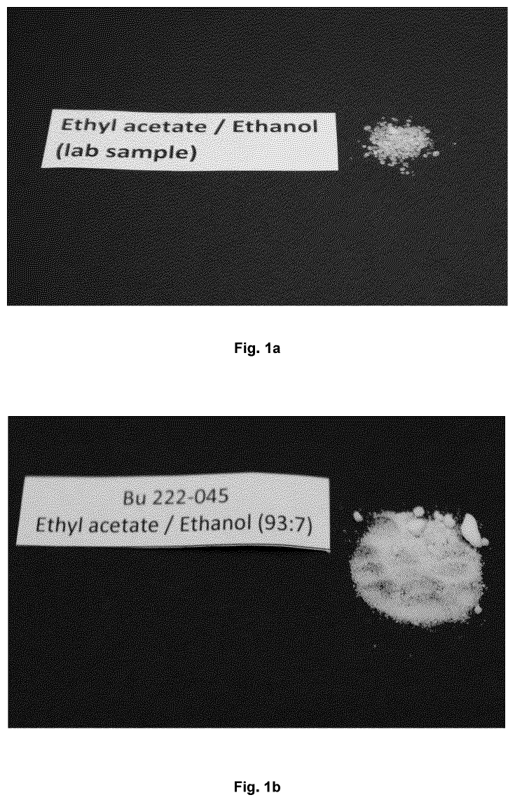

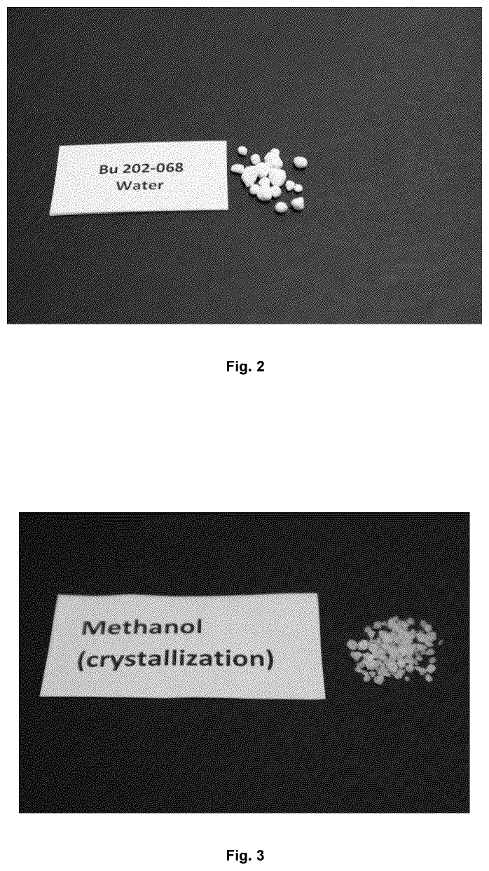

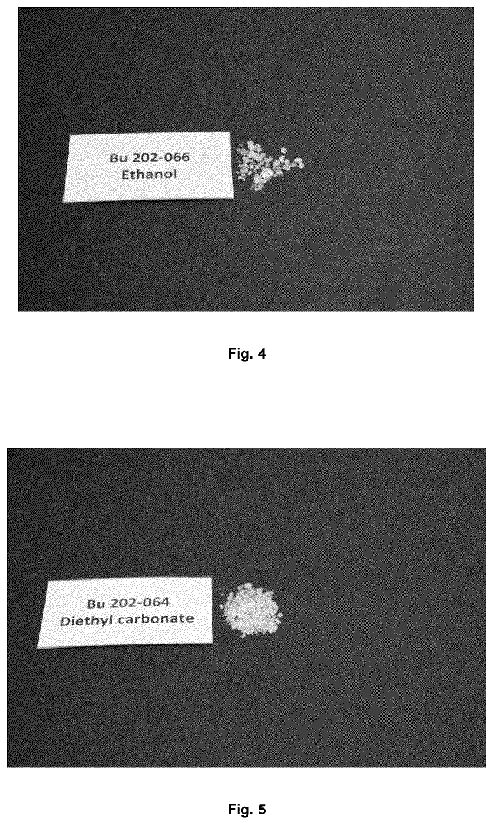

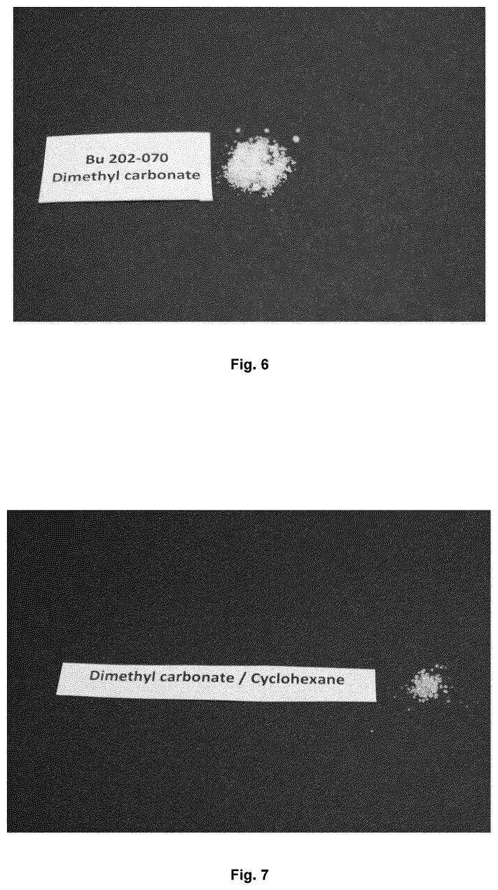

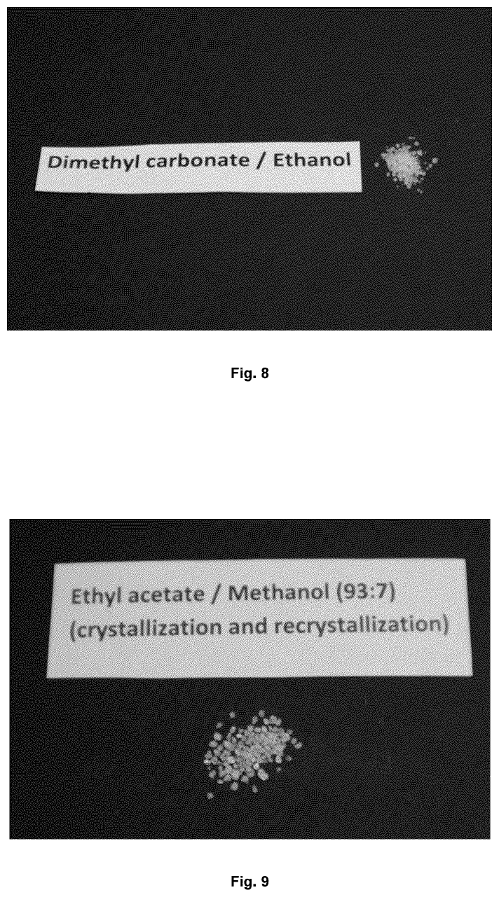

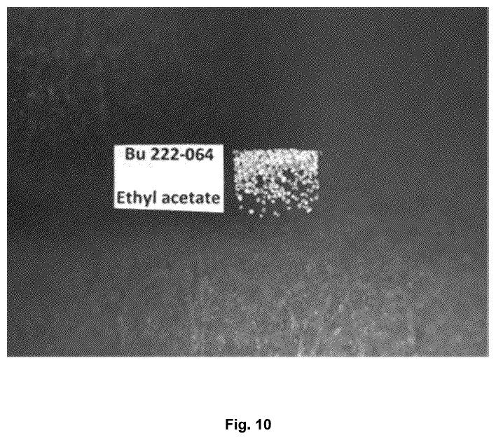

2.1 4-Hydroxyacetophenone prepared according to example 1a (solvent: ethanol/ethyl acetate; according to the invention): See FIG. 1a
2.2 4-Hydroxyacetophenone prepared according to example 1b (solvent system: ethanol/ethyl acetate; according to the invention): See FIG. 1b
2.3 4-Hydroxyacetophenone prepared according to example 2 (solvent: water; not according to the invention): See FIG. 2
2.4 4-Hydroxyacetophenone prepared according to example 3 (solvent: methanol; not according to the invention): See FIG. 3
2.5 4-Hydroxyacetophenone prepared according to example 4 (solvent: ethanol; not according to the invention): See FIG. 4
2.6 4-Hydroxyacetophenone prepared according to example 5 (solvent: diethyl carbonate; not according to the invention): See FIG. 5
2.7 4-Hydroxyacetophenone prepared according to example 6 (solvent: dimethyl carbonate; not according to the invention): See FIG. 6
2.8 4-Hydroxyacetophenone prepared according to example 7 (solvent: dimethyl carbonate/cyclohexane; not according to the invention): See FIG. 7
2.9 4-Hydroxyacetophenone prepared according to example 8 (solvent: dimethyl carbonate/ethanol; not according to the invention): See FIG. 8
2.10 4-Hydroxyacetophenone prepared according to example 9 (solvent: methanol/ethyl acetate; not according to the invention): See FIG. 9
2.11 4-Hydroxyacetophenone prepared according to example 10 (solvent: ethyl acetate; not according to the invention): See FIG. 10

TABLE 1
Physical appearance of the samples of purified 4-hydroxyacetophenone

| 4-HAP prepared | | |
|---|---|---|
| according to | Solvent(s) used for | |
| example | purification of 4-HAP | Physical appearance of 4-HAP |
| 1a (according to | ethanol/ethyl acetate | Uniform, small crystals; white color |
| invention) | | |
| 1b (according to | ethanol/ethyl acetate | Uniform, small crystals; white color |
| invention) | | |
| 2 (comparative) | Water | Globules; large crystals; pale pink |
| | | color |
| 3 (comparative) | Methanol | Crystal structure more rough; slight |
| | | yellowish discoloration |
| 4 (comparative) | Ethanol | Rough crystal structure; crystal sizes |
| | | not homogenous; white color |
| 5 (comparative) | Diethyl carbonate | Uniform, small crystals; white color |
| 6 (comparative) | Dimethyl carbonate | Uniform, small crystals; white color |
| 7 (comparative) | Dimethyl carbonate/ | Uniform, small crystals; white color |
| | cyclohexane | |
| 8 (comparative) | Dimethyl carbonate/ | Uniform, small crystals; pale white |
| | ethanol | color |
| 9 (comparative) | Methanol/ethyl acetate | Rough crystal structure; white color |
| 10 (comparative) | Ethyl acetate | Uniform, medium crystals; white color |

Purified 4-hydroxyacetophenone obtained by the method according to the invention has uniform, small crystals, which ensure good flowability and are easy to work with for a formulator. It also has a desirable white color.

### 3 Solubility Testing of the Different Samples of Purified 4-Hydroxyacetophenone (0.5 wt.-%) in Cold Water

### Method:

Make sure that clumps are broken up and the purified sample of 4-hydroxyacetophenone is uniformly free-flowing, but do not mill or grind the purified 4-hydroxyacetophenone
Provide water as solvent at a temperature of 17.5-18.5° C.
Add full amount of sample of purified 4-hydroxyacetophenone to the provided cold solvent at once (cf. Table 2 below for concentrations used)
Stir at 500 rpm on magnetic stirrer (without heating) at 20-21° C.
Record time from the addition of the sample of the purified 4-hydroxyacetophenone to the provided solvent until all of the purified 4-hydroxyacetophenone is dissolved

TABLE 2
Formulations for and results of solubility testing in cold water

| | | | Time needed |
|---|---|---|---|
| 4-HAP purified | Solvent(s) used for | | until purified |
| according to | purification of | Materials used in | 4-HAP is fully |
| example | 4-HAP | solubility testing | dissolved |
| 1a (according | ethanol/ethyl | 0.5 wt.-% 4-HAP | 24 minutes |
| to invention) | acetate | 99.50 wt.-% water | |
| 1b (according | ethanol/ethyl | 0.5 wt.-% 4-HAP | 22 minutes |
| to invention) | acetate | 99.50 wt.-% water | |
| 2 (comparative) | Water | 0.5 wt.-% 4-HAP | 115 minutes |
| | | 99.50 wt.-% water | |
| 3 (comparative) | Methanol | 0.5 wt.-% 4-HAP | 35 minutes |
| | | 99.50 wt.-% water | |
| 4 (comparative) | Ethanol | 0.5 wt.-% 4-HAP | 41 minutes |
| | | 99.50 wt.-% water | |
| 5 (comparative) | Diethyl carbonate | 0.5 wt.-% 4-HAP | 35 minutes |
| | | 99.50 wt.-% water | |
| 6 (comparative) | Dimethyl carbonate | 0.5 wt.-% 4-HAP | 51 minutes |
| | | 99.50 wt.-% water | |
| 7 (comparative) | Dimethyl carbonate/ | 0.5 wt.-% 4-HAP | 51 minutes |
| | cyclohexane | 99.50 wt.-% water | |
| 8 (comparative) | Dimethyl carbonate/ | 0.5 wt.-% 4-HAP | 44 minutes |
| | ethanol | 99.50 wt.-% water | |
| 9 (comparative) | Methanol/ethyl | 0.5 wt.-% 4-HAP | 29 minutes |
| | acetate | 99.50 wt.-% water | |
| 10 (comparative) | Ethyl acetate | 0.5 wt.-% 4-HAP | 67 minutes |
| | | 99.50 wt.-% water | |

The purified 4-hydroxyacetophenone obtained according to examples 1a and 1b surprisingly showed by far the shortest dissolution time in cold water.

### 4. Solubility Testing of the Different Samples of Purified 4-Hydroxyacetophenone (0.5 wt.-%) in Cold 1,2-Pentanediol (5 wt.-%) in Water

### Method:

Make sure that clumps are broken up and the purified sample of 4-hydroxyacetophenone is uniformly free-flowing, but do not mill or grind the purified 4-hydroxyacetophenone
Provide 5 wt.-% of 1,2-pentanediol in water as solvent at a temperature of 17.5-18.5° C.
Add full amount of sample of purified 4-hydroxyacetophenone to the provided cold solvent at once (cf. Table 3 below for concentrations used)
Stir at 500 rpm on magnetic stirrer (without heating) at 20-21° C.
Record time from the addition of the sample of the purified 4-hydroxyacetophenone to the solvent until all of the purified 4-hydroxyacetophenone is dissolved

TABLE 3
Formulations for and results of solubility testing in cold
1,2-pentanediol (5 wt.-%) in water

| | | | Time needed |
|---|---|---|---|
| | Solvent(s) | | until purified |
| 4-HAP purified | used for | Materials used in | 4-HAP is fully |
| according to | purification | solubility testing | dissolved |
| 1a (according | ethanol/ | 0.5 wt.-% 4-HAP | 12 minutes |
| to invention) | ethyl | 5 wt.-% 1,2-pentanediol | |
| | acetate | 94.50 wt.-% water | |
| 2 (comparative) | Water | 0.5 wt.-% 4-HAP | 56 minutes |
| | | 5 wt.-% 1,2-pentanediol | |
| | | 94.50 wt.-% water | |
| 3 (comparative) | Methanol | 0.5 wt.-% 4-HAP | 22 minutes |
| | | 5 wt.-% 1,2-pentanediol | |
| | | 94.50 wt.-% water | |
| 4 (comparative) | Ethanol | 0.5 wt.-% 4-HAP | 33 minutes |
| | | 5 wt.-% 1,2-pentanediol | |
| | | 94.50 wt.-% water | |
| 5 (comparative) | Diethyl | 0.5 wt.-% 4-HAP | 26 minutes |
| | carbonate | 5 wt.-% 1,2-pentanediol | |
| | | 94.50 wt.-% water | |
| 6 (comparative) | Dimethyl | 0.5 wt.-% 4-HAP | 35 minutes |
| | carbonate | 5 wt.-% 1,2-pentanediol | |
| | | 94.50 wt.-% water | |
| 7 (comparative) | Dimethyl | 0.5 wt.-% 4-HAP | 36 minutes |
| | carbonate/ | 5 wt.-% 1,2-pentanediol | |
| | cyclohexane | 94.50 wt.-% water | |
| 8 (comparative) | Dimethyl | 0.5 wt.-% 4-HAP | 29 minutes |
| | carbonate/ | 5 wt.-% 1,2-pentanediol | |
| | ethanol | 94.50 wt.-% water | |
| 9 (comparative) | Methanol/ | 0.5 wt.-% 4-HAP | 17 minutes |
| | ethyl | 5 wt.-% 1,2-pentanediol | |
| | acetate | 94.50 wt.-% water | |

The purified 4-hydroxyacetophenone obtained according to example 1a surprisingly showed by far the shortest dissolution time in cold 1,2-pentanediol (5 wt.-%) in water.

### 5. Solubility Testing of the Different Samples of Purified 4-Hydroxyacetophenone (0.5 wt.-%) in Cold 1,2-Pentanediol (3 wt.-%) and Glycerin (5 wt.-%) in Water

### Method:

Make sure that clumps are broken up and the purified sample of 4-hydroxyacetophenone is uniformly free-flowing, but do not mill or grind the purified 4-hydroxyacetophenone
Provide 3 wt.-% of 1,2-pentanediol and 5 wt.-% glycerin in water as solvent at a temperature of 17.5-18.5° C.
Add full amount of sample of purified 4-hydroxyacetophenone to the provided cold solvent at once (cf. Table 4 below for concentrations used)
Stir at 500 rpm on magnetic stirrer (without heating) at 20-21° C.
Record time from the addition of the sample of the purified 4-hydroxyacetophenone to the solvent until all of the purified 4-hydroxyacetophenone is dissolved

TABLE 4
Formulations for and results of solubility testing in cold
1,2-pentanediol (3 wt.-%) and glycerin (5 wt.-%) in water

| | Solvent(s) | | Time needed |
|---|---|---|---|
| 4-HAP purified | used for | | until purified |
| according to | purification | Materials used in | 4-HAP is fully |
| example | of 4-HAP | solubility testing | dissolved |
| 1a (according | ethanol/ | 0.5 wt.-% 4-HAP | 15 minutes |
| to invention) | ethyl | 3 wt.-% 1,2-pentanediol | |
| | acetate | 5 wt.-% glycerin (99.5%) | |
| | | 91.50 wt.-% water | |
| 1b (according | ethanol/ | 0.5 wt.-% 4-HAP | 19 minutes |
| to invention) | ethyl | 3 wt.-% 1,2-pentanediol | |
| | acetate | 5 wt.-% glycerin (99.5%) | |
| | | 91.50 wt.-% water | |
| 2 (comparative) | Water | 0.5 wt.-% 4-HAP | 53 minutes |
| | | 3 wt.-% 1,2-pentanediol | |
| | | 5 wt.-% glycerin (99.5%) | |
| | | 91.50 wt.-% water | |
| 3 (comparative) | Methanol | 0.5 wt.-% 4-HAP | 30 minutes |
| | | 3 wt.-% 1,2-pentanediol | |
| | | 5 wt.-% glycerin (99.5%) | |
| | | 91.50 wt.-% water | |
| 4 (comparative) | Ethanol | 0.5 wt.-% 4-HAP | 24 minutes |
| | | 3 wt.-% 1,2-pentanediol | |
| | | 5 wt.-% glycerin (99.5%) | |
| | | 91.50 wt.-% water | |
| 5 (comparative) | Diethyl | 0.5 wt.-% 4-HAP | 22 minutes |
| | carbonate | 3 wt.-% 1,2-pentanediol | |
| | | 5 wt.-% glycerin (99.5%) | |
| | | 91.50 wt.-% water | |
| 6 (comparative) | Dimethyl | 0.5 wt.-% 4-HAP | 28 minutes |
| | carbonate | 3 wt.-% 1,2-pentanediol | |
| | | 5 wt.-% glycerin (99.5%) | |
| | | 91.50 wt.-% water | |
| 7 (comparative) | Dimethyl | 0.5 wt.-% 4-HAP | 33 minutes |
| | carbonate/ | 3 wt.-% 1,2-pentanediol | |
| | cyclohexane | 5 wt.-% glycerin (99.5%) | |
| | | 91.50 wt.-% water | |
| 8 (comparative) | Dimethyl | 0.5 wt.-% 4-HAP | 27 minutes |
| | carbonate/ | 3 wt.-% 1,2-pentanediol | |
| | ethanol | 5 wt.-% glycerin (99.5%) | |
| | | 91.50 wt.-% water | |
| 9 (comparative) | Methanol/ | 0.5 wt.-% 4-HAP | 21 minutes |
| | ethyl | 3 wt.-% 1,2-pentanediol | |
| | acetate | 5 wt.-% glycerin (99.5%) | |
| | | 91.50 wt.-% water | |
| 10 (comparative) | Ethyl | 0.5 wt.-% 4-HAP | 42 minutes |
| | acetate | 3 wt.-% 1,2-pentanediol | |
| | | 5 wt.-% glycerin (99.5%) | |
| | | 91.50 wt.-% water | |

The purified 4-hydroxyacetophenone obtained according to examples 1a and 1b surprisingly showed by far the shortest dissolution time in cold 1,2-pentanediol (3 wt.-%) and glycerin (5 wt.-%) in water.

The examples above demonstrate that 4-hydroxyacetophenone purified by the method according to the invention has strongly superior solubility properties in a range of standard solvent systems for cosmetic formulations.

1-15. (canceled)

16. A method for purifying crude 4-hydroxyacetophenone comprising:
(a) providing crude 4-hydroxyacetophenone,
(b) mixing the crude 4-hydroxyacetophenone with a combination of ethanol and ethyl acetate to form a mixture,
(c) optionally, heating the mixture and dissolving the crude 4-hydroxyacetophenone,
(d) optionally, adding an adsorbent to the mixture,
(e) optionally, cooling the mixture to a temperature above crystallization temperature of 4-hydroxyacetophenone,
(f) if the adsorbent was added to the mixture in (d), removing the adsorbent from the mixture,
(g) cooling the mixture to a temperature below the crystallization temperature of 4-hydroxyacetophenone to induce crystallization of the 4-hydroxyacetophenone,
(h) collecting the crystallized 4-hydroxyacetophenone of (g), and optionally, carrying out (i) to (k) one or more times:
(i) dissolving the crystallized 4-hydroxyacetophenone of (h) or (k) to form a solution in a combination of ethanol and ethyl acetate, optionally with heating,
(j) cooling the solution to a temperature below the crystallization temperature of the 4-hydroxyacetophenone to induce crystallization of 4-hydroxyacetophenone,
(k) collecting the crystallized 4-hydroxyacetophenone,
(l) optionally, drying the crystallized 4-hydroxyacetophenone obtained in (h) or (k).

17. The method of claim 16, wherein the combination of the ethanol and the ethyl acetate in (b) and/or (i) is 0.01 to 50 wt. % of ethanol in ethyl acetate.

18. The method of claim 16, wherein 0.1 to 80 wt. % of the 4-hydroxyacetophenone is mixed with the combination of the ethanol and the ethyl acetate in (b) and/or (i), based on a total weight of the mixture or solution in (b) and/or (i).

19. The method of claim 16, wherein the mixture obtained in (b) is heated to a reflux temperature of at least 30° C. and maintained at the reflux temperature for at least 1 minute.

20. The method of claim 16 comprising adding 0.1 to 25 wt. % of the adsorbent of (d) to the mixture, based on a total weight of the mixture.

21. The method of claim 16 comprising cooling the mixture to a temperature of 25 to 75° C. in (e) and/or (g).

22. The method of claim 16 comprising cooling the mixture to a temperature of −10° C. to below room temperature in (e) and/or (g).

23. The method of claim 16 comprising cooling the solution to a temperature of −10° C. to below room temperature in (j).

24. The method of claim 16 comprising (I) drying the crystallized 4-hydroxyacetophenone at reduced pressure of 0.1 to 100 mbar.

25. The method of claim 16 comprising (I) drying the crystallized 4-hydroxyacetophenone at a temperature of 50 to 100° C.

26. The method of claim 16 comprising (I) drying the crystallized 4-hydroxyacetophenone for 1 to 48 hours.

27. A product comprising the crystallized 4-hydroxyacetophenone and the combination of the ethanol and the ethyl acetate obtained or obtainable by the method of claim 16.

28. The product of claim 27, wherein the combination of the ethanol and the ethyl acetate in the product is less than 10000 ppm, based on a total weight of the product.

29. The product of claim 27, wherein the combination of the ethanol and the ethyl acetate in the product is less than 5000 ppm, based on a total weight of the product.

30. The method of claim 16 comprising (d) adding the adsorbent, wherein the adsorbent is activated carbon.

31. The method of claim 16 comprising (d) adding the adsorbent, and the adsorbent is removed by filtration in (f).

32. The method of claim 16 comprising (I) drying the crystallized 4-hydroxyacetophenone obtained in (h) or (k) until residual ethanol and ethyl acetate in the 4-hydroxyacetophenone is less than 10000 ppm.

33. The method of claim 16 comprising (I) drying of the crystallized 4-hydroxyacetophenone obtained in (h) or (k) until residual ethanol and ethyl acetate in the 4-hydroxyacetophenone is less than 5000 ppm.
